(19) United States
(12) Patent Application Publication
(10) Pub. No.: US 2023/0007845 A1
(43) Pub. Date: Jan. 12, 2023

(54) Submersion-Free Systems and Methods to Genetically Transform Cannabis Sativa

(71) Applicant: Rev Genomics, Inc., Oakland, CA (US)

(72) Inventors: Phillip Adam Conklin, Oakland, CA (US); Julie Ann Pham, Oakland, CA (US)

(21) Appl. No.: 17/860,013

(22) Filed: Jul. 7, 2022

Related U.S. Application Data
(60) Provisional application No. 63/219,743, filed on Jul. 8, 2021.

(51) Int. Cl. C12N 15/82

(52) U.S. Cl. CPC C12N 15/8205

(57) ABSTRACT

Exemplary embodiments include methods for improved transformation and shoot induction of Cannabis sativa, including culturing a Cannabis sativa cotyledon explant utilizing a transformation method, and co-cultivating the Cannabis sativa cotyledon explant with Agrobacterium. The transformation methods do not include liquid media.

### CROSS-REFERENCE TO RELATED APPLICATIONS

This non-provisional application claims the priority benefit of U.S. Provisional Application Ser. No. 63/219,743, filed on Jul. 8, 2021 titled “Submersion-Free Methods to Genetically Transform Cannabis sativa,” which is hereby incorporated by reference herein in its entirety, including all appendices and all references cited therein.

### FIELD OF INVENTION

The present technology relates to efficient genetic systems and methods for transforming Cannabis sativa.

### SUMMARY

Exemplary embodiments include methods for improved transformation and shoot induction of Cannabis sativa, including culturing a Cannabis sativa cotyledon explant utilizing a transformation method, and co-cultivating the Cannabis sativa cotyledon explant with Agrobacterium. The transformation method does not include liquid media.

In further exemplary embodiments, a regeneration method is used. The regeneration method does not include liquid media. The methods include gently rubbing a surface of the Cannabis sativa cotyledon explant against a lawn of the Agrobacterium. In various exemplary embodiments, a proximal adaxial region of the Cannabis sativa cotyledon explant with the lawn of the Agrobacterium is not in contact with media during co-cultivation. The transformation method may be a biotic transformation method, or a abiotic transformation method.

The Cannabis sativa cotyledon explant, in some exemplary embodiments, may be partially recalcitrant to a transformation protocol that utilizes liquid media, or may be totally recalcitrant to a transformation protocol that utilizes liquid media. The Cannabis sativa cotyledon explant may be partially recalcitrant to a regeneration protocol that utilizes liquid media, or the Cannabis sativa cotyledon explant may be totally recalcitrant to a regeneration protocol that utilizes liquid media. Additionally, methods may include gently rubbing a surface of the Cannabis cotyledon explant against an Agrobacterium coated tool.

The Cannabis cotyledon explant, in various exemplary embodiments may regenerate a shoot, and in some cases, regenerating a shoot on a proximal adaxial surface of the Cannabis cotyledon explant. Additionally, the Cannabis cotyledon explant may regenerate a shoot after 15 days on a solid media. The Cannabis cotyledon explant may regenerate a shoot on a proximal adaxial surface of the Cannabis cotyledon explant after 15 days on a solid media and/or the Cannabis cotyledon explant may regenerate a plurality of shoots after 15 days on a solid media. In various exemplary cases, the Cannabis cotyledon explant may regenerate a plurality of shoots after 15 days on a solid media at a frequency as high as 88.9%. The Cannabis cotyledon explant may also regenerate a shoot on a proximal adaxial surface of the Cannabis cotyledon explant after 3 days of co-cultivation with the Agrobacterium.

### DESCRIPTION OF THE DRAWINGS

Certain embodiments of the present technology are illustrated by the accompanying figures. It will be understood that the figures are not necessarily to scale. It will be understood that the technology is not necessarily limited to the particular embodiments illustrated herein.

FIG. 1 shows lack of shoot regeneration using Cannabis cotyledon explants after 15 days on several types of liquid media. The lower right shows the development of callus, but no shoots on an explant.

FIG. 2 shows shoot regeneration using Cannabis cotyledon explants after 15 days on several types of solid media. The lower right shows the development of shoots on the proximal adaxial surface of an explant.

TABLE 1 shows shoot regeneration frequency of explants after 15 days on several different types of solid or liquid media.

FIG. 3 shows the transformation rate of the proximal adaxial surface after infection of explants with Agrobacterium. (Black) Submerged for 1 hour in a liquid Agrobacterium culture or (Gray) after the proximal adaxial surface of the explant has rubbed against a lawn of Agrobacterium. Transformation rates of the proximal adaxial surface were counted after 3 days of co-cultivation with Agrobacterium.

FIG. 4 shows transformed fluorescent cells on the proximal adaxial surface of a Cannabis cotyledon explant after 3 days of co-cultivation with Agrobacterium.

FIG. 5 shows the transformation rate of the proximal adaxial surface after co-cultivation of explants. (Black) Proximal adaxial surface down and in contact with the media or (Gray) Proximal adaxial surface up and suspended in air away from the media. Transformation rates of the proximal adaxial surface were calculated after 3 days of co-cultivation with Agrobacterium.

FIG. 6 shows exemplary embodiments of fluorescent shoots. (Left) Brightfield, (Middle) DSRed filter, (Right) GFP filter.

### DETAILED DESCRIPTION

The detailed embodiments of the present technology are disclosed here. It should be understood, that the disclosed embodiments are merely exemplary of the technology, which may be embodied in multiple forms. Those details disclosed herein are not to be interpreted in any form as limiting, but as the basis for the claims.

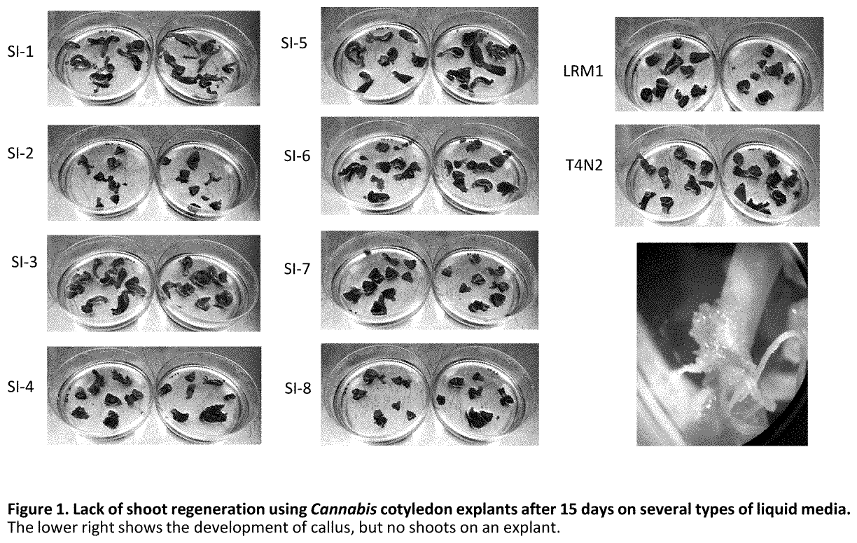

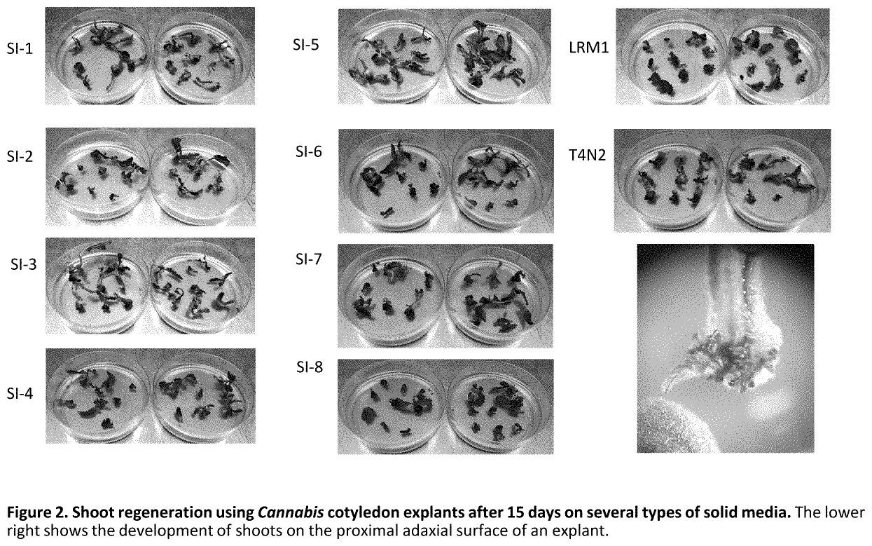

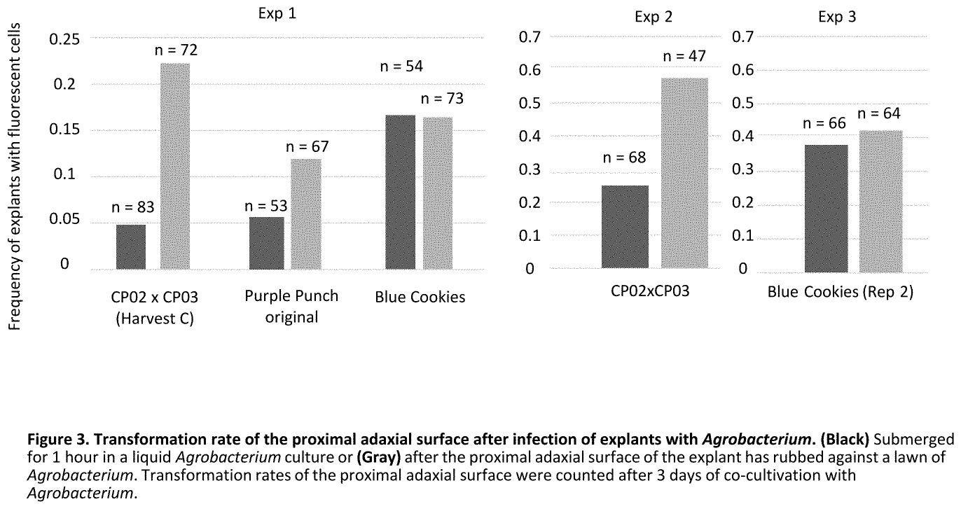

Cannabis sativa is a high value dicotyledonous crop bred as marijuana for its medicinal use or hemp for its fiber. Therefore, the use of advanced breeding techniques, like genetic transformation, to increase yield are highly desirable and yet difficult to achieve. The exemplary embodiments herein include methods to genetically transform Cannabis by avoiding submersion in liquid. These methods overcome the inhibition that liquid submersion and culture has on Cannabis shoot regeneration as evidenced in FIG. 1 and TABLE 1, so the use of a submersion-free Agrobacterium mediated transformation is preferred when regenerating transformed Cannabis tissue as evidenced in FIG. 2 and TABLE 1. This is unexpected since Humulus lupulus, another member of the Cannabaceae family, makes use of liquid culture for efficient shoot regeneration. See e.g., Batista, Dora, et al. “Adventitious shoot mass production of hop (Humulus lupulus L.) var. Eroica in liquid medium from organogenic nodule cultures.” Plant Science 151.1 (2000): 47-57.

The use of submersion-free Agrobacterium mediated infection.

Exemplary embodiments herein include the submersion-free application of Agrobacterium to the proximal adaxial region of the Cannabis cotyledon explant, which is the region capable of regenerating shoots. Exemplary methods include use of several well-formed colonies of Agrobacterium, with a construct of interest, suspended in 100 microliters (“ul”) of Lennox LB Broth (“LB”) and spread evenly with an L-shaped cell spreader on LB selection plates. The cotyledons held by their distal ends, are gently rubbed against a small population of Agrobacterium with the proximal adaxial region. These exemplary methods promote a higher rate and quality of transformation as well an increase in the rate of shoot induction after transformation. Most Agrobacterium mediated transformation protocols require explants incubate in liquid infection media with Agrobacterium at a specified optical density. The exemplary methods herein show submersion-free Agrobacterium mediated transformation increases the transformation rate of Cannabis cotyledon explants by 1 to 4.5 fold, as evidenced in FIG. 2. In some genotypes, the transformation rate is equivalent to submersion methods, suggesting these exemplary methods improve upon genotypes recalcitrant to submersion methods. Some exemplary embodiments include a dry-rub with Agrobacterium coated tools (i.e. scalpel), or with other organisms that can genetically transform plants.

The cotyledon's proximal adaxial surface suspended in air during co-cultivation.

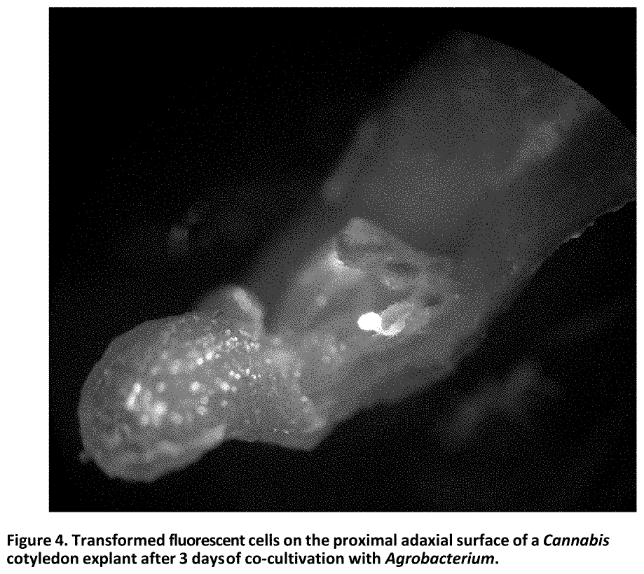

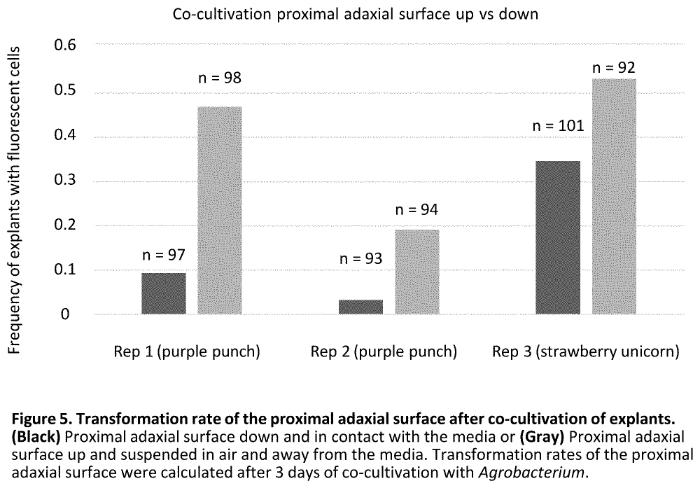

Exemplary embodiments include the orientation of the Cannabis cotyledon explant proximal adaxial surface up or having the distal end embedded in the media such that the proximal adaxial surface is not in contact with media during co-cultivation. This is used to promote Agrobacterium mediated transformation in Cannabis within the proximal adaxial surface, which is the region capable of shoot regeneration as evidenced by FIG. 4. Many transformation protocols require the co-cultivation of cotyledon explants after infection with Agrobacterium. These protocols also require the cotyledon to be placed adaxial side down or submerged in media during co-cultivation, or adaxial side up. However, Cannabis is unique in that a co-cultivation with a cotyledon oriented with the proximal adaxial surface face up and suspended in air can increase in the rate of transformation of the explant by 1.5 to 6 fold as evidenced by FIG. 5, compared to one that has the proximal adaxial surface down and in contact with media.

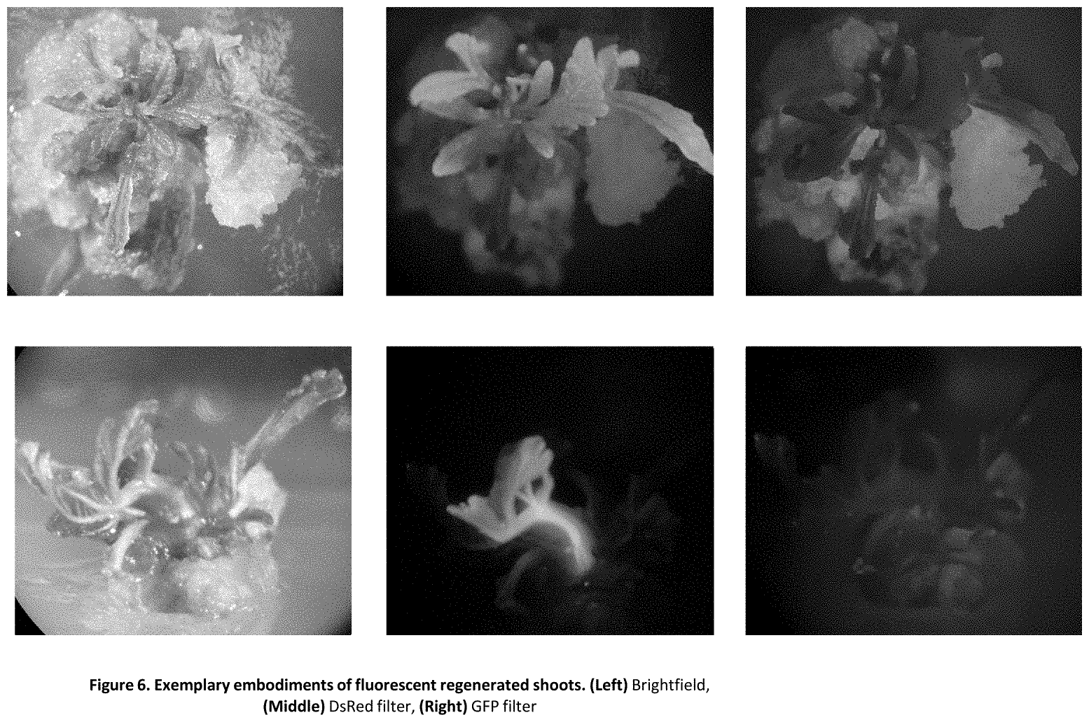

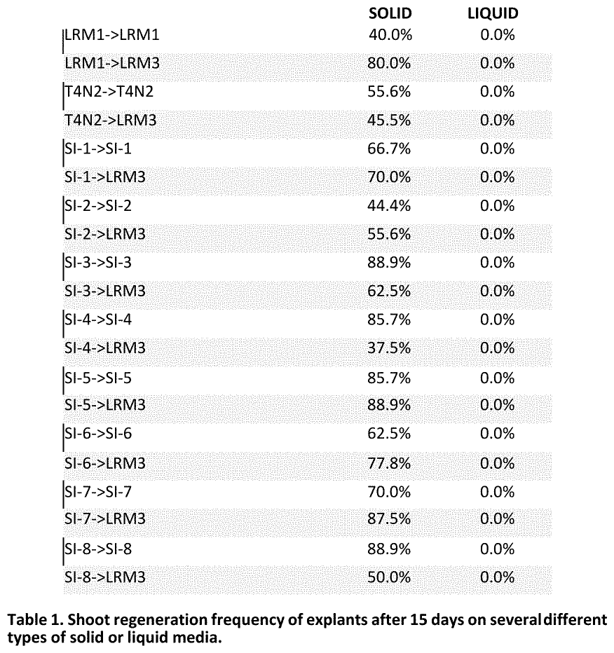

Exemplary embodiments of genetically transformed Cannabis shoots can be seen FIG. 6.

What is claimed is:

1. A method for improved transformation and shoot induction of Cannabis sativa, the method comprising:
culturing a Cannabis sativa cotyledon explant utilizing a transformation method, and
co-cultivating the Cannabis sativa cotyledon explant with Agrobacterium.

2. The method of claim 1, wherein the transformation method does not include liquid media.

3. The method of claim 1, further comprising using a regeneration method.

4. The method of claim 4, wherein the regeneration method does not include liquid media.

5. The method of claim 1, further comprising gently rubbing a surface of the Cannabis sativa cotyledon explant against a lawn of the Agrobacterium.

6. The method of claim 5, further comprising:
ensuring a proximal adaxial region of the Cannabis sativa cotyledon explant with the lawn of the Agrobacterium is not in contact with media during co-cultivation.

7. The method of claim 1, wherein the transformation method is a biotic transformation method.

8. The method of claim 1, wherein the transformation method is a abiotic transformation method.

9. The method of claim 1, wherein the Cannabis sativa cotyledon explant is partially recalcitrant to a transformation protocol that utilizes liquid media.

10. The method of claim 1, wherein the Cannabis sativa cotyledon explant is totally recalcitrant to a transformation protocol that utilizes liquid media.

11. The method of claim 1, wherein the Cannabis sativa cotyledon explant is partially recalcitrant to a regeneration protocol that utilizes liquid media.

12. The method of claim 1, wherein the Cannabis sativa cotyledon explant is totally recalcitrant to a regeneration protocol that utilizes liquid media.

13. The method of claim 1, further comprising gently rubbing a surface of the Cannabis cotyledon explant against an Agrobacterium coated tool.

14. The method of claim 1, further comprising the Cannabis cotyledon explant regenerating a shoot.

15. The method of claim 1, further comprising the Cannabis cotyledon explant regenerating a shoot on a proximal adaxial surface of the Cannabis cotyledon explant.

16. The method of claim 1, further comprising the Cannabis cotyledon explant regenerating a shoot after 15 days on a solid media.

17. The method of claim 1, further comprising the Cannabis cotyledon explant regenerating a shoot on a proximal adaxial surface of the Cannabis cotyledon explant after 15 days on a solid media.

18. The method of claim 1, further comprising the Cannabis cotyledon explant regenerating a plurality of shoots after 15 days on a solid media.

19. The method of claim 1, further comprising the Cannabis cotyledon explant regenerating a plurality of shoots after 15 days on a solid media at a frequency as high as 88.9%.

20. The method of claim 1, further comprising the Cannabis cotyledon explant regenerating a shoot on a proximal adaxial surface of the Cannabis cotyledon explant after 3 days of co-cultivation with the Agrobacterium.
